(19) United States
(12) Patent Application Publication
(10) Pub. No.: US 2015/0366852 A1
(43) Pub. Date: Dec. 24, 2015

(54) Prevention of Obesity in Antipsychotic, Antidepressant and Antiepileptic Medication

(71) Applicant: N-Gene Research Laboratories, Inc., New York, NY (US)

(72) Inventors: Peter Literati Nagy, Budapest (HU); Jesse Roth, Whitestone, NY (US); Zoltan Szilvassy, Debrecen-Jozsa (HU); Kalman Tory, Budapest (HU); Michael Brownstein, Rockville, MD (US); Kálmán Takács, Budapest (HU); Laszlo Vigh, Szeged (HU); József Mandl, Budapest (HU); Balázs Sümegi, Pecs (HU); Sandor Bernath, Teiki (HU); Attila Kolonics, Budapest (HU); Gábor Balogh, Szeged (HU); Janos Egri, Budapest (HU)

(21) Appl. No.: 14/748,150

(22) Filed: Jun. 23, 2015

Related U.S. Application Data
(63) Continuation of application No. 11/687,954, filed on Mar. 19, 2007, now abandoned.
(60) Provisional application No. 60/856,177, filed on Nov. 2, 2006.

(51) Int. Cl. A61K 31/4545; A61K 45/06

(52) U.S. Cl. CPC A61K 31/4545; A61K 45/06

(57) ABSTRACT

O-(3-piperidino-2-hydroxypropyl)-nicotinic amidoxime or a pharmaceutically suitable acid addition salt thereof (BGP-15) can be used for the prevention or reduction of weight gain or obesity in a patient treated with an antipsychotic drug or an antidepressant drug or an antiepileptic drug.

### RELATED APPLICATION INFORMATION

This application is a continuation and claims priority to U.S. application Ser. No. 11/687,954, filed Mar. 19, 2007, which claims priority to U.S. Provisional Application Ser. No. 60/856,177, filed Nov. 2, 2006.

### BACKGROUND

Anti-psychotic drugs are used for the treatment of psychiatric disorders, particularly schizophrenia, while anti-depressants are administered to alleviate the symptoms of depression. Many patients treated with anti-psychotics, e.g., olanzapine or clozapine feel acoria due to a failure in the regulation of food uptake, thus, the treatment frequently leads to weight gain. Overweight and even obesity may occur within 3-6 months after the beginning of the treatment as evidenced by reports on treated patients [Blin, Can. J. Psychiatry 44:235-44 (1999)]. In a similar manner, medication with many antidepressants e.g. amitriptyline, imipramine etc. or antiepileptics (anticonvulsants) e.g. valproic acid, sodium valproate etc. results in weight gain that may lead to obesity [Ruetsch et al., L'Encéphale 31:507-16 (2005)]. Overweight and obesity themselves are associated with hypertension and abnormal metabolic changes such as insulin resistance and dyslipidemia which are risk factors for diabetes. Obesity (particularly abdominal obesity), insulin resistance and dyslipidemia are major features of “pre-diabetes” (metabolic syndrome) that leads to type 2 diabetes mellitus. Diabetes is associated with serious complications such as retinopathy, nephropathy, and neuropathy. In addition, diabetes is accompanied by increased mortality due to a greater risk of cardiovascular disease.

Due to the extensive and growing administration of antipsychotic and antiepileptic drugs and antidepressants to patients in the United States and throughout the developed countries, the above undesirable side effects are considered as an increasing problem which is related to increased rates of mortality and morbidity. In addition, patients requiring a treatment with an antipsychotic or an antidepressant or an antiepileptic may decide to stop treatment because of the induced weight gain.

O-(3-piperidino-2-hydroxy-1-propyl)-nicotinic amidoxime) (abbreviated as BGP-15) was patented in 1976 as a new compound useful in the treatment of diabetic angiopathy, a complication of diabetes resulting in the damage of blood vessels (see, e.g., U.S. Pat. No. 4,187,220). BGP-15 has the structure depicted below.

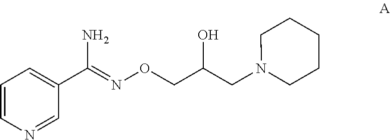 [SMILES: N/C(=N\OCC(O)CN1CCCCC1)c1cccnc1]

U.S. Pat. No. 6,306,878 refers to a method for the protection of the mitochondrial genome and/or mitochondrion from damage leading to myopathies and neurodegenerative diseases which comprises administering an effective non-toxic dose to a patient susceptible to such damage of an amidoximic acid derivative including BGP-15. A preferred myopathy is cardiomyopathy. Neurodegenerative diseases include Parkinson's disease, Huntington's disease and amyotrophic lateral sclerosis.

U.S. Pat. No. 6,458,371 refers to a composition comprising 0.1-30% of a hydroximic acid derivative including BGP-15 as the active ingredient and a carrier that is in the form of a cream, lotion, foam or spray. The composition is suitable for reducing the incidence of photodamage by radiation with UV-B.

U.S. Pat. No. 6,884,424 refers to a method for preventing actinic keratosis by applying a hydroximic acid derivative including BGP-15 to the affected skin surface.

U.S. Pat. No. 6,451,851 refers to a method of treating a patient suffering from a viral infection comprising administering to the patient a pharmaceutically effective amount of a known antivirally active agent together with a hydroximic acid derivative including BGP-15.

U.S. Pat. No. 6,440,998 refers to a pharmaceutical composition having antitumor activity with reduced side effect comprising cisplatin or carboplatin and a hydroximic acid derivative including BGP-15.

U.S. Pat. No. 6,656,955 refers to a pharmaceutical composition having antitumor activity with reduced side effect comprising paclitaxel or docetaxel and a hydroximic acid derivative including BGP-15.

U.S. Pat. No. 6,720,337 refers to a pharmaceutical composition having antitumor activity with reduced side effect comprising oxaliplatin and a hydroximic acid derivative including BGP-15.

U.S. Pat. No. 6,838,469 refers to a pharmaceutical composition having antitumor activity with reduced side effect comprising pyrimidine derivatives and BGP-15.

PCT Patent Application WO 00/07580 disclosed experimental data for the antidiabetic effect of BGP-15 in the treatment of type 1 diabetes mellitus. It is to be noted that type 1 diabetes mellitus is an autoimmune disease occurring at young age, while type 2 diabetes mellitus is a metabolic disease occurring at higher age.

PCT Application WO 03/007951 refers to a pharmaceutical combination of hydroximic acid derivatives including BGP-15 and an antidiabetic or anti-hyperlipidemic active agent for the prevention or treatment of a prediabetic state, metabolic X-syndrome or diabetes mellitus as well as disorders which are associated with the states listed above, namely endogenic metabolic disorders, insulin resistance, dislipidemia, alopecia, diffuse effluvium and/or female endocrine disorders based on androgenic preponderance. In the description, laboratory data indicate that BGP-15 enhances, synergistically, the effect of the known antidiabetic agent metformin and troglitazone, respectively. The laboratory data also show that BGP-15 in itself enhances the insulin sensitivity (thus, reduces the insulin resistance) in both normal and hyper-cholesterolemic animals relative to the control.

PCT Application WO 2005/122678 refers to the use of BGP-15 in a pharmaceutical composition having prokinetic effect (i.e. induces activity in the stomach and intestines. Prokinetic effect includes possible treatment of reflux esophagitis, gastroparesis, influencing bile flow from the gall bladder etc.

PCT Application WO 2005/123049 refers to the use of BGP-15 for mitochondrial genesis i.e. to increase the number of mitochondria in the cells resulting in a roborating effect.

PCT Application WO 2006/079910 refers to the use of BGP-15 for the treatment of lesions in the oral cavity, especially periodontal disease.

### SUMMARY

It has been found that O-(3-piperidino-2-hydroxypropyl)-nicotinic amidoxime or a pharmaceutically suitable acid addition salt thereof (BGP-15) can be used for the prevention or reduction of weight gain or obesity in a patient treated with an antipsychotic drug or an antidepressant drug or an antiepileptic drug.

Described herein are methods for preventing or reducing the side effect leading to weight gain or obesity in a patient requiring a treatment with an antipsychotic or antidepressant or antiepileptic drug comprising administering an effective amount of a known antipsychotic or antidepressant or antiepileptic and an effective non-toxic amount of O-(3-piperidino-2-hydroxy-1-propyl)-nicotinic amidoxime or a pharmaceutically acceptable acid addition salt thereof to the patient, wherein the administration of the O-(3-piperidino-2-hydroxy-1-propyl)-nicotinic amidoxime or a pharmaceutically acceptable acid addition salt thereof prevents or reduces the metabolic side-effect experienced by the patient requiring treatment with an antipsychotic or antidepressant or antiepileptic drug.

Also described is a pharmaceutical composition having antipsychotic or antidepressant or antiepileptic activity with reduced side effect comprising a known antipsychotic or antidepressant or antiepileptic and O-(3-piperidino-2-hydroxy-1-propyl)-nicotinic amidoxime or a pharmaceutically acceptable acid addition salt thereof in admixture with one or more conventional carrier(s).

In various embodiments: the antipsychotic agent is selected from the group consisting of olanzapine, clozapine, risperidone, quetiapine and sulpiride; and O-(3-piperidino-2-hydroxy-1-propyl)-nicotinic amidoxime dihydrochloride is administered.

Also described is a pharmaceutical composition having antipsychotic or antidepressant or antiepiliptic activity with reduced side effect comprising a known antipsychotic or antidepressant and O-(3-piperidino-2-hydroxy-1-propyl)-nicotinic amidoxime or a pharmaceutically acceptable acid addition salt thereof in admixture with one or more conventional carrier(s). In various embodiments: the known antipsychotic agent is selected from the group consisting of olanzapine, clozapine, risperidone, quetiapine and sulpiride; the composition comprises olanzapine and O-(3-piperidino-2-hydroxy-1-propyl)-nicotinic amidoxime or a pharmaceutically acceptable acid addition salt thereof and the composition comprises clozapine and O-(3-piperidino-2-hydroxy-1-propyl)-nicotinic amidoxime or a pharmaceutically acceptable acid addition salt thereof.

Also described is a method for treating a patient being treated with an antidepressant medication or antipsychotic medication, the method comprising administering to the patient being treated with an antidepressant or antipsychotic or antiepileptic medication, a composition comprising O-(3-piperidino-2-hydroxy-1-propyl)-nicotinic amidoxime or a pharmaceutically acceptable acid addition salt thereof to the patient. In various embodiments: the patient is being treated with an antipsychotic medication, the antipsychotic medication is an atypical antipsychotic medication; the antipsychotic medicine causes weight gain in at least some patient; the antipsychotic medication is selected from the group consisting of: olanzapine, clozapine, risperidone, quetiapine, sulpiride, ziprasidone, aripiprazole, sertindole, zotepine, amisulpride and N-desmethylclozapine; the pharmaceutically acceptable salt of O-(3-piperidino-2-hydroxy-1-propyl)-nicotinic amidoxime is O-(3-piperidino-2-hydroxy-1-propyl)-nicotinic amidoxime dihydrochloride; the medication is an antidepressant; the patient has suffered weight gain after being administered the antipsychotic or antidepressant medication; the patient has a body mass index greater than 25 kg/m²; the patient has a body mass index greater than 30 kg/m²; and the antipsychotic medication is olanzapine, risperidone or clozapine.

Also described herein are pharmaceutical compositions comprising an antipsychotic or antidepressant or antiepileptic medication and O-(3-piperidino-2-hydroxy-1-propyl)-nicotinic amidoxime or a pharmaceutically acceptable acid addition salt thereof. In various embodiments: the antipsychotic medication is selected from the group consisting of: olanzapine, clozapine, risperidone, quetiapine and sulpiride, ziprasidone, and aripiprazole; the antipsychotic medication is selected from the group consisting of: olanzapine, riperidone and clozapine.

Also described is packaging containing a first pharmaceutical composition comprising an antipsychotic medication or an antidepressant medication or an antiepileptic medication and a second pharmaceutical composition comprising O-(3-piperidino-2-hydroxy-1-propyl)-nicotinic amidoxime or a pharmaceutically acceptable acid addition salt thereof. In various embodiments: the antipsychotic medication is selected from the group consisting of: olanzapine, clozapine, risperidone, quetiapine and sulpiride. Also described as packaging containing a unit dosage formulation comprising O-(3-piperidino-2-hydroxy-1-propyl)-nicotinic amidoxime or a pharmaceutically acceptable acid addition salt thereof and a unit dosage formulation of antipsychotic medication or an antidepressant medication or an antiepileptic medication.

Also described is a pharmaceutical composition having antipsychotic or antidepressant or antiepileptic activity with reduced side effect comprising a known antipsychotic or antidepressant or antiepileptic and O-(3-piperidino-2-hydroxy-1-propyl)-nicotinic amidoxime or a pharmaceutically acceptable acid addition salt thereof in admixture with one or more conventional carrier(s).

The details of one or more embodiments of the invention are set forth in the accompanying drawings and the description below. Other features, objects, and advantages of the invention will be apparent from the description and drawings, and from the claims.

### DETAILED DESCRIPTION

An antipsychotic drug is a drug used to treat severe mental disorders (psychoses) including schizophrenia and mania as well as certain other conditions. Some antipsychotic agents are administered in small doses to relieve anxiety.

One useful group of antipsychotic drugs include phenothiazine derivatives of formula IA

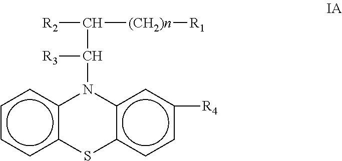

and pharmaceutically suitable acid addition salts thereof, wherein
   R₁ represents a di(C₁₋₄ alkyl)amino, 1-(C₁₋₄ alkyl)piperidyl, 4-(C₁₋₄ alkyl)piperazinyl or 4-[2-hydroxy(C₁₋₄ alkyl)]-1-piperazinyl group,
   R₂ and R₃ stand, independently, for a hydrogen atom or C₁₋₄ alkyl group,
   R₄ means a hydrogen or halo atom, carboxy, C₁₋₄ alkoxy, C₁₋₄ alkanoyl, trifluoromethyl, methylmercapto or methylsulfinyl group, and
   n has a value of 0 or 1.

In certain embodiments: R₁ represents a dimethylamino, 1-methylpiperidyl, 4-methylpiperazinyl or 4-(2-hydroxyethyl)-1-piperazinyl group,

R₂ and R₃ stand, independently, for a hydrogen atom or methyl group,
R₄ means a hydrogen or chloro atom, carboxy, methoxy, acetyl, trifluoromethyl, methylmercapto or methylsulfinyl group, and
n has a value of 0 or 1.

Among the compound having formula IA are: chlorpromazine (2-chloro-N,N-dimethyl-10H-phenothiazine-10-propanamine), promazine (N,N-dimethyl-10H-phenothiazine-10-propanamine), mesoridazine (10-[2-(1-methyl-2-piperidinyl)ethyl]-2-(methylsulfinyl)-10H-phenothiazine), fluphenazine (4-[3-[2-(trifluoromethyl)-10H-phenothiazin-10-yl]propyl]-1-piperazineethanol), and trifluoperazine (10-[3-(4-methyl-1-piperazinyl)propyl]-2-(trifluoromethyl)-10H-phenothiazine), as well as pharmaceutically suitable acid addition salts thereof.

Another useful group of antipsychotics include thioxanthene derivatives of formula IB

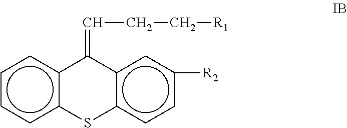

and pharmaceutically suitable acid addition salts thereof, wherein
R₁ represent a di(C₁₋₄ alkyl)amino, 4-(C₁₋₄ alkyl)-1-piperazinyl, 4-[2-hydroxy(C₁₋₄ alkyl)]-1-piperazinyl, 4-[2-(C₁₋₄ alkanoyloxy)-(C₁₋₄ alkyl)]-1-piperazinyl or 4-(2-decanoyloxyethyl)-1-piperazinyl group,
R₂ stands for a halo atom, trifluoromethyl or N,N-dimethylsulfonylamido group.

In various embodiments of the compound of formula IB

R₁ represent a dimethylamino, 4-methyl-1-piperazinyl, 4-(2-hydroxyethyl)-1-piperazinyl, 4-(2-acetoxyethyl)-1-piperazinyl or 4-(2-decanoyloxyethyl)-1-piperazinyl group,
R₂ stands for a chloro atom, trifluoromethyl or N,N-dimethylsulfonylamido group.

Among the useful compounds having formula IB are: chlorprothixene (3-(2-chloro-9H-thioxanthen-9-ylidene)-N,N-dimethyl-1-propanamine), clopenthixol (4-[3-(2-chloro-9H-thioxanthen-9-ylidene)propyl]-1-piperazine-ethanol), thiothixene (N,N-dimethyl-9-[3-(4-methyl-1-piperazinyl)propylidene]-thioxanthene-2-sulfonamide), and flupentixol (4-[3-(2-(trifluoromethyl)-9H-thioxanthen-9-ylidene)propyl]-1-piperazine-ethanol), as well as pharmaceutically suitable acid addition salts thereof.

Additional useful antipsychotics include compounds of formula IC:

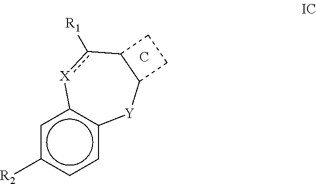

wherein
   X stands for a nitrogen atom or a group of formula —C═ or —CH—,
   Y represents a group of formula —NH—, oxygen or nitrogen atom,
   R₁ means a 4-(2-hydroxyethoxyethyl)-1-piperazinyl, 4-(C₁₋₄ alkyl)-1-piperazinyl or 4-(3-hydroxypropyl)-1-piperazinyl group,
   R₂ is a hydrogen or halo atom,
   ring C represents a benzene ring optionally substituted by a halo atom or N,N-dimethylsulfonamido group or ring C stands for a heterocyclic group that forms with the benzodiazepine portion a thieno[2,3-b][1,5]benzodiazepine structure, wherein the 5-membered thieno ring is optionally substituted in position 2 by a methyl group,
   the dotted line between X and the adjacent carbon atom has no meaning in case of the saturated ring, otherwise the dotted line represents a valence bond,
   and, if chemically possible, pharmaceutically suitable acid addition salts thereof.

In certain embodiments of the compounds of formula IC:

R₁ means a 4-(2-hydroxyethoxyethyl)-1-piperazinyl, 4-(methyl)-1-piperazinyl or 4-(3-hydroxypropyl)-1-piperazinyl group,
R₂ is a hydrogen or chloro atom, and
X, Y, ring C and the dotted line are as defined above.

Among the useful compounds having formula IC are: clozapine (8-chloro-11-(4-methyl-1-piperazinyl)-5H-dibenzo[b,e][1,4]diazepine), olanzapine (2-methyl-4-(4-methyl-1-piperazinyl)-10H-thieno[2,3-b][1,5]-benzodiazepine), quetiapine (2-[2-(4-dibenzo[b,f][1,4]thiazepin-11-yl-1-piperazinyl)ethoxy]-ethanol), zotepine (2-[(8-chlorodibenzo[b,f]thiepin-10-yl)oxy]-N,N-dimethylethanamine), isoclozapine (chloro-11-(4-methyl-1-piperazinyl)-5H-dibenzo[b,e][1,4]-diazepine), clothiapine (2-chloro-11-(4-methyl-1-piperazinyl)dibenzo[b,f][1,4]thiazepine), oxithepine (10-[4-(3-hydroxypropyl)piperazino]-10,11-dihydrodibenzo[b,f]-thiepine), and, if chemically possible, pharmaceutically suitable acid addition salts thereof.

Additional useful antipsychotics include benzamide derivatives of formula ID

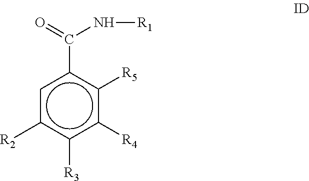

and pharmaceutically suitable acid addition salts thereof, wherein
R₁ represents an N-[1-(C₁₋₄ alkyl)-2-pyrrolidinyl]-(C₁₋₄ alkyl), 2-[di(C₁₋₄ alkyl)-amino]-(C₁₋₄ alkyl) or 1-benzyl-3-pyrrolidinyl group,
R₂ stands for a hydrogen or halo atom, aminosulfonyl or (C₁₋₄ alkyl)sulfonyl group,
R₃ means a hydrogen or halo atom, amino or (C₁₋₄ alkyl)amino group,
R₄ is hydrogen or halo atom or methoxy group,
R₅ represents a C₁₋₄ alkoxy or allyloxy group.

In some embodiments: R₁ represents an N-(1-ethyl-2-pyrrolidinyl)methyl, 2-(diethylamino)ethyl or 1-benzyl-3-pyrrolidinyl group,

R₂ stands for a hydrogen or chloro atom, aminosulfonyl or ethylsulfonyl group,
R₃ means a hydrogen or chloro atom, amino or methylamino group,
R₄ is hydrogen or bromo atom or methoxy group.

Among the useful antipsychotics having formula ID are: sulpiride (5-(aminosulfonyl)-N-[(1-ethyl-2-pyrrolidinyl)methyl]-2-methoxy-benzamide), amisulpride (4-amino-N-[(1-ethyl-2-pyrrolidinyl)methyl]-5-(ethylsulfonyl)-2-methoxybenzamide) and remoxipride ((S)-3-bromo-N-[(1-ethyl-2-pyrrolidinyl)-methyl]-2,6-dimethoxybenzamide), as well as pharmaceutically suitable acid addition salts thereof.

Additional useful antipsychotics consists of the benzisoxazole derivatives of formula IF

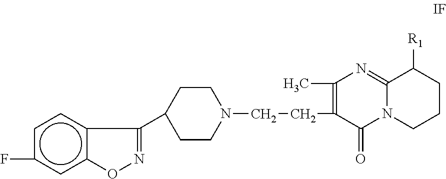

wherein
R₁ represents a hydrogen atom or hydroxy group.

Among the useful benzisoxazole derivatives of formula IF are: risperidone (3-[2-[4-(6-fluoro-1,2-benzisoxazol-3-yl)-1-piperidinyl]ethyl]-6,7,8,9-tetrahydro-2-methyl-4H-pyrido[1,2-a]pyrimidin-4-one) and paliperidone (3-[2-[4-(6-fluoro-1,2-benzisoxazol-3-yl)-1-piperidinylethyl]-6,7,8,9-tetrahydro-9-hydroxy-2-methyl-4H-pyrido[1,2-c]pyrimidin-4-one) and suitable salts thereof.

Additional useful antipsychotics include diphenylbutyl-piperidine derivatives of formula IG

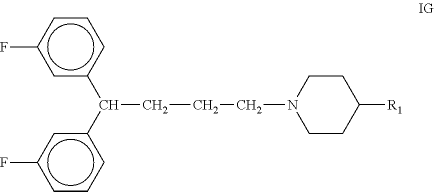

wherein
R₁ represents a 2-benzimidazolon-1-yl group.

Among the useful compounds having formula IG is: pimozide (1-[1-[4,4-bis(4-fluorophenyl)butyl]-4-piperidinyl]-1,3-dihydro-2H-benzimidazol-2-one).

Additional useful antipsychotics include butyrophenone derivatives and pharmaceutically suitable acid addition salts thereof such as the following compounds: haloperidol i.e. 4-[4-(4-chlorophenyl)-4-hydroxy-1-piperidinyl]-1-(4-fluorophenyl)-1-butanone, bromperidol i.e. 4-[4-(4-bromophenyl)-4-hydroxy-1-piperidinyl]-1-(4-fluorophenyl)-1-butanone or trifluperidol i.e. 1-(4-fluorophenyl)-4-[4-hydroxy-4-[3-(trifluoromethyl)phenyl]-1-piperidinyl]-1-butanone.

Additional antipsychotics include indole derivatives and pharmaceutically suitable acid addition salts thereof such as the following compounds: molindone (3-ethyl-1,5,6,7-tetrahydro-2-methyl-5-(4-morpholinylmethyl)-4H-indol-4-one), ziprasidone (5-[2-[4-(1,2-benzisothiazol-3-yl)-1-piperazinyl]ethyl]-6-chloro-1,3-dihydro-2H-indol-2-one), sertindole (1-[2-[4-[5-chloro-1-(4-fluorophenyl)-1H-indol-3-yl]-1-piperidinyl]-ethyl]-2-imidazolidinone) and oxypertine (5,6-dimethoxy-2-methyl-3-[2-(4-phenyl-1-piperazinyl)ethyl]-1H-indole).

The term antidepressant refers to a drug that alleviates the symptoms of depression. A preferred group of antidepressants includes bicyclic compounds such as paroxetine ((3S-trans)-3-[(1,3-benzodioxol-5-yloxy)methyl]-4-(4-fluorophenyl)-piperidine) and pharmaceutically suitable acid addition salts thereof.

Another useful group of antidepressants include tricyclic compounds such as amitriptyline (3-(10,11-dihydro-5H-dibenzo[a,d]cyclohepten-5-ylidene)-N,N-dimethyl-1-propanamine), doxepin (3-dibenz[b,e]oxepin-11(6H)ylidene-N,N-dimethyl-propanamine), imipramine (10,11-dihydro-N,N-dimethyl-5H-dibenz[b,f]azepine-5-propan-amine), clomipramine (3-chloro-10,11-dihydro-N,N-dimethyl-5H-dibenz[b,f]azepine-5-propanamine), nortriptyline (3-(10,11-dihydro-5H-dibenzo[a,d]cyclohepten-5-ylidene)-N-methyl-1-propanamine), trimipramine (10,11-dihydro-N,N,β-trimethyl-5H-dibenz[b,f]azepine-5-propanamine), and desipramine (10,11-dihydro-N-methyl-5H-dibenz[b,f]azepine-5-propanamine), as well as pharmaceutically suitable acid addition salts thereof.

A further useful group of antidepressants includes tetracyclic compounds such as maprotiline (N-methyl-9,10-ethanoanthracene-9(10H)-propanamine) and pharmaceutically suitable acid addition salts thereof.

The term “antiepileptic” or “anticonvulsant” refers to a drug that prevents or reduces the severity and frequency of seizures in various types of epilepsy. A preferred group of antiepileptics includes certain phenothiazine derivatives of formula IA such as triflupromazine (N,N-dimethyl-2-(trifluoromethyl)-10H-phenothiazine-10-propanamine) and metofenazate (3,4,5-trimethoxybenzoic acid 2-[4-[3-(2-chloro-10H-phenothiazin-10-yl)propyl]-1-piperazinyl]ethyl ester) which latter is typically administered as the difumarate. Said phenothiazine derivatives possess, in addition to antipsychotic, also antiepileptic activity.

A further preferred group of antiepileptics includes benzodiazepine derivatives such as clonazepam (5-(2-chlorophenyl)-1,3-dihydro-7-nitro-2H-1,4-benzodiazepine-2-one), clobazam (7-chloro-1-methyl-5-phenyl-1H-1,5-benzodiazepine-2,4(3H,5H)-dione) etc., dibenzazepine derivatives such as carbamazepine (5H-dibenz[b,f]azepine-5-carboxamide) having also analgesic activity, oxcarbazepine (10,11-dihydro-10-oxo-5H-dibenz[b,f]azepine-5-carboxamide) etc., barbituric acid derivatives having also hypnotic and sedative activity such as phenobarbital (5-ethyl-5-phenyl-2,4,6(1H,3H,5H)-pyrimidine-trione) and pharmaceutically suitable metal salts thereof, eterobarb (5-ethyl-1,3-bis(methoxy-methyl)-5-phenyl-2,4,6(1H,3H,5H)-pyrimidinetrione), proxibarbal (5-(2-hydroxy-propyl)-5-(2-propenyl)-2,4,6(1H,3H,5H)-pyrimidinetrione), primidone (5-ethyl-dihydro-5-phenyl-4,6(1H,5H)-pyrimidinedione) etc., hydantoin derivatives such as phenytoin (5,5-diphenyl-2,4-imidazolidinedione), mephenytoin (5-ethyl-3-methyl-5-phenyl-2,4-imidazolidinedione), fosphenytoin (5,5-diphenyl-3-phosphonoyl-methyl-2,4-imidazolidinedione) etc. and pharmaceutically suitable metal salts thereof, oxazolidine derivatives such as ethadione (3-ethyl-5,5-dimethyl-2,4-oxazolidinedione) etc., succinimide derivatives such as ethosuximide (3-ethyl-3-methyl-2,5-pyrrolidine-dione), phensuximide (1-methyl-3-phenyl-2,5-pyrrolidinedione) etc., carboxylic acid derivatives such as valproic acid (2-propylpentanoic acid) and pharmaceutically suitable metal salts thereof, valpromide (2-propylpentanamide), valnoctamide (2-ethyl-3-methyl-pentanamide) etc.

An additional useful group of antiepileptics includes gamma-aminobutyric acid (GABA) derivatives such as gabapentin (1-(aminomethyl)cyclohexaneacetic acid), progabide (4-[[(4-chlorophenyl)(5-fluoro-2-hydroxyphenyl)methylene]-amino]butanamide), vigabatrin (4-amino-5-hexenoic acid), piracetam (2-oxo-1-pyrrolidineacetamide), oxiracetam (4-hydroxy-2-oxo-1-pyrrolidineacetamide), nefiracetam (N-(2,6-dimethylphenyl)-2-oxo-1-pyrrolidineacetamide) etc. and pharmaceutically suitable metal salts of the acids, carbamate derivatives such as meprobamate (2-methyl-2-propyl-1,3-propanediol dicarbamate) having also anxiolytic effect, felbamate (2-phenyl-1,3-propanediol dicarbamate) etc., some sulfonamides such as acetazolamide (N-[5-(aminosulfonyl)-1,3,4-thiadiazol-2-yl]acetamide), zonisamide (1,2-benzisoxazole-3-methanesulfonamide), sulthiame (4-(tetrahydro-2H-1,2-thiazin-2-yl)-benzenesulfonamide S,S-dioxide) etc., N-acylurea derivatives such as phenacemide (N-(aminocarbonyl)benzene-acetamide), pheneturide (N-(aminocarbonyl)-α-ethylbenzeneacetamide) etc.

Additional useful antiepileptics include lamotrigine (6-(2,3-dichlorophenyl)-1,2,4-triazine-3,5-diamine), topiramate (2,3:4,5-bis-O-(1-methylethylidene)-β-D-fructopyranose sulfamate), and tiagabine ((R)-1-[4,4-bis(3-methyl-2-thienyl)-3-butenyl]-3-piperidinecarboxylic acid) and pharmaceutically suitable metal salts thereof.

An especially preferred group of antiepileptics includes valproic acid and pharmaceutically suitable alkali metal valproates.

A pharmaceutically suitable acid addition salt is a salt formed with an inorganic acid such as hydrochloric acid, sulfuric acid etc. or with an organic acid such as acetic acid, lactic acid, tartaric acid etc. Useful acid addition salts include hydrochlorides, acetates, maleates etc. A preferred acid addition salt of O-(3-piperidino-2-hydroxy-1-propyl)nicotinic amidoxime is the dihydrochloride thereof.

In the context of the description and claims, the expression “metabolic side-effect” corresponds to the side-effect experienced in antipsychotic, antidepressant or antiepileptic medication which leads to weight gain, overweight or obesity.

BGP-15 can be prepared by the process described in, e.g., U.S. Pat. No. 4,187,220.

In one embodiment, a conventional dose of a known antipsychotic or antidepressant or antiepileptic drug is administered to a patient requiring treatment with an antipsychotic or antidepressant or antiepileptic drug, and, simultaneously, a dose of BGP-15 or a pharmaceutically suitable acid addition salt thereof is administered. This non-toxic dose of BGP-15 prevents or reduces, effectively, the weight gain associated with the administration of the antipsychotic or antidepressant or antiepileptic drug leading otherwise to overweight or even obesity. In some embodiments, the antipsychotic medication or the antidepressant medication or the antiepileptic medication is not administered simultaneously with BGP-15. Thus, while the two or more agents in the combination therapy, e.g., BGP-15 and olanzapine, can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other. In some cases even longer intervals are possible. While in many cases it is desirable that the two or more agents used in a combination therapy be present in within the patient's body at the same time, this need not be so. Combination therapy can also include two or more administrations of one or more of the agents used in the combination. For example, if agent X and agent Y are used in a combination, one could administer them sequentially in any combination one or more times, e.g., in the order X—Y—X, X—X—Y, Y—X—Y, Y—Y—X, X—X—Y—Y, etc.

Combination therapy can also include the administration of two or more agents via different routes or locations. For example, (a) one agent is administered orally and another agents is administered intravenously or (b) one agent is administered orally and another is administered locally. In each case, the agents can either simultaneously or sequentially.

Generally, the daily dose of antipsychotic, antidepressant or antiepileptic drugs for an adult person of about 70 kg body weight amounts to 1-1000 mg. The similar daily dose of BGP-15 (as dihydrochloride) is, in general, 5-1000 mg, preferably 50-500 mg.

According to certain embodiments, 10-20 mg of olanzapine or 100-800 mg of clozapine and 50-500 mg of BGP-15 dihydrochloride are administered to an adult, daily.

In case of pharmaceutical compositions either or both of the two active agents (i.e. the known antipsychotic or antidepressant or antiepileptic drug and BGP-15) has been converted, one by one, to separate pharmaceutical compositions using one or more conventional carrier(s) and any of the usual processes of drug manufacture, and in this case the two sorts of pharmaceutical composition obtained are administered to the patient simultaneously or one after the other.

Alternatively, the two active agents have been converted to one single pharmaceutical composition that can be administered to the patient being in need thereof. In the latter case, the pharmaceutical composition may contain a mixture of the two active agents, or each of the active agents may be present at a different site in the pharmaceutical composition, e.g., one of them in the tablet core and the other in a coating of the tablet core. Of course, one or more conventional carriers and any of the usual processes of drug manufacture are used to prepare this single pharmaceutical composition.

The pharmaceutical compositions described herein contain an effective non-toxic amount of an antipsychotic or antidepressant or antiepileptic drug or a pharmaceutically suitable acid addition salt or metal salt thereof and an effective non-toxic amount of BGP-15 or a pharmaceutically suitable acid addition salt thereof in addition to one or more pharmaceutically acceptable carrier(s). The pharmaceutical composition may include any dosage form suitable for peroral, parenteral or rectal administration or for local treatment, and can be solid or liquid.

In principle, the pharmaceutical composition of the invention may contain more then one antipsychotic, antidepressant and/or antiepileptic drug.

The solid pharmaceutical compositions suitable for peroral administration may be powders, capsules, tablets, film-coated tablets, microcapsules etc., and can comprise binding agents such as gelatine, sorbitol, poly(vinylpyrrolidone) etc.; filling agents such as lactose, glucose, starch, calcium phosphate etc.; auxiliary substances for tabletting such as magnesium stearate, talc, poly(ethylene glycol), silica etc.; wetting agents such as sodium laurylsulfate etc. as the carrier.

The liquid pharmaceutical compositions suitable for peroral administration may be solutions, suspensions or emulsions and can comprise, e.g., suspending agents such as gelatine, carboxymethylcellulose etc.; emulsifiers such as sorbitane monooleate etc.; solvents such as water, oils, glycerol, propylene glycol, ethanol etc.; preservatives such as methyl p-hydroxybenzoate etc. as the carrier.

Pharmaceutical compositions suitable for parenteral administration consist of sterile solutions of the active ingredients, in general.

Dosage forms listed above as well as other dosage forms are known per se, see e.g. Remington's Pharmaceutical Sciences, 18th Edition, Mack Publishing Co., Easton, USA (1990).

The pharmaceutical composition contains dosage unit, in general. The daily dose can be administered in one or more portions. The actual dosage depends on many factors and is determined by the doctor.

The pharmaceutical composition is prepared by admixing the active ingredients to one or more carrier(s), and converting the mixture obtained to a pharmaceutical composition in a manner known per se. Useful methods are known from the literature, e.g. Remington's Pharmaceutical Sciences mentioned above.

In some embodiments the pharmaceutical composition contains an antipsychotic drug selected from the group consisting of olanzapine, clozapine, risperidone, quetiapine and sulpiride in addition to BGP-15 or a pharmaceutically suitable acid addition salt thereof.

### Example 1

### Effect of BGP-15 on the Body Weight Gain Induced by Olanzapine in Rats

Groups of female Wistar rats were treated with vehicle (control group) and the compounds to be tested for 28 days. Each group consisted of 6 animals fed with normal laboratory chow and tap water ad libitum. The compounds to be tested were administered twice daily, at 8 h and 18 h, perorally. The antipsychotic olanzapine was administered in a dose of 1 mg/kg to induce body weight gain. BGP-15 was administered in a dose of 10 mg/kg, alone and together with olanzapine. The oral antidiabetics metformin (100 mg/kg) and rosiglitazone (3 mg/kg) were employed as reference compounds, alone and together with olanzapine. The average starting weight of the animals was 171 g. The weights of the animals at the end of the test on the 28^{th} day are listed in Table 1.

TABLE 1

| | Body weight (average in the |
|---|---|
| Treatment | group) in g |
| Control | 255 |
| Olanzapine, 1 mg/kg | 330 |
| BGP-15 dihydrochloride, 10 mg/kg | 242 |
| Metformin, 100 mg/kg | 266 |
| Rosiglitazone, 3 mg/kg | 284 |
| Olanzapine, 1 mg/kg + | 262 |
| BGP-15 dihydrochloride, 10 mg/kg | |
| Olanzapine, 1 mg/kg + | 331 |
| metformin, 100 mg/kg | |
| Olanzapine, 1 mg/kg + | 359 |
| rosiglitazone, 3 mg/kg | |

The weight gain of the control group relative to the starting weight during the test period of 28 days can be considered as normal in case of rats. The group treated with olanzapine had a significantly greater average weight than the control group. This is consistent with the obesity inducing effect of olanzapine observed in patients treated with this drug. Treatment with BGP-15 alone produced somewhat lower average weight, while treatment with metformin and rosiglitazone, respectively, produced somewhat higher average weight relative to the control group. Treatment with metformin did not reduce, while treatment with rosiglitazone increased the weight gain induced by olanzapine. However, treatment with BGP-15 dihydrochloride prevented the weight gain induced by olanzapine.

### Example 2

### Effect of BGP-15 on the Body Weight Gain Induced by Olanzapine or Clozapine in Mice

Groups of female NMRI mice were treated with vehicle (control group) and the compounds to be tested for 15 days, perorally. Each group consisted of 10 animals fed with normal laboratory chow and tap water ad libitum. Treatments were performed between 5 and 6 pm, shortly before the dark phase, the primary feeding period of the day. The antipsychotic olanzapine was administered in a dose of 0.5 mg/kg, while the antipsychotic clozapine was administered in a dose of 1 mg/kg to induce body weight gain. BGP-15 was administered in a dose of 10 mg/kg, alone and together with olanzapine and clozapine, respectively. The weights of the animals were recorded twice weekly and the change in the body weights of the animals between the first and 15^{th} days are given in Table 2.

TABLE 2

| | Body weight gain (average |
|---|---|
| Treatment | in the group) in g |
| Control | 2.98 |
| Olanzapine, 0.5 mg/kg | 3.5 |
| Clozapine, 1 mg/kg | 4.11 |
| BGP-15 dihydrochloride, 10 mg/kg | 2.85 |
| Olanzapine, 0.5 mg/kg + | 2.33 |
| BGP-15 dihydrochloride, 10 mg/kg | |
| Clozapine, 1 mg/kg + | 2.19 |
| BGP-15 dihydrochloride, 10 mg/kg | |

Both antipsychotic drugs caused increased body weight gain relative to the control group. BGP-15 alone reduced body weight gain somewhat. In combination with the antipsychotic drugs, BGP-15 prevented the weight increasing side effect thereof and even further reduced the body weight change relative to the control group.

### Example 3

### Effect of BGP-15 on the Body Weight Gain Induced by Risperidone in Rats

The experiments were carried out in eight-week-old female Wistar rats. Each test group consisted of 10 animals fed with normal laboratory chow and tap water ad libitum. The animals were treated with vehicle (control group) and the compounds to be tested for 21 days. The antipsychotic risperidone was injected subcutaneously once daily in doses of 0.005 and 0.05 mg/kg, respectively to induce body weight gain. BGP-15 dihydrochloride was administered in a dose of 20 mg/kg, perorally, once daily, alone and together with risperidone.

The average starting weight of the animals was 195 g. The weight gains of the animals at the end of the test on the 21^{st} day are listed in Table 3

TABLE 3

| Treatment | Body weight gain (g) |
|---|---|
| Control | 27 |
| BGP-15 dihydrochloride 20 mg/kg p.o. | 22.7 |
| Risperidone 0.005 mg/kg s.c. | 39.7 |
| Risperidone 0.05 mg/kg s.c. | 41 |
| Risperidone 0.005 mg/kg s.c. + | 25.8 |
| BGP-15 dihydrochloride 20 mg/kg p.o. | |
| Risperidone 0.05 mg/kg s.c. + | 28.7 |
| BGP-15 dihydrochloride 20 mg/kg p.o. | |

Both doses of the antipsychotic drug risperidone caused increased body weight gain relative to the control group. BGP-15 alone reduced body weight gain somewhat. In combination with the antipsychotic drug, BGP-15 prevented the weight increasing side effect thereof in both doses.

Thus, the above tests indicate that BGP-15 can effectively reduce the weight gain induced by antipsychotics, while the known oral antidiabetic drugs having also insulin sensitizing effect metformin and rosiglitazone used as reference agents were of no useful effect. Consequently, BGP-15 can be used to effectively prevent or reduce weight gain, overweight or obesity.

A number of embodiments of the invention have been described. Nevertheless, it will be understood that various modifications may be made without departing from the spirit and scope of the invention. Accordingly, other embodiments are within the scope of the following claims.

What is claimed is:

1. A method for preventing or reducing the side effect leading to weight gain or obesity in a patient requiring a treatment with an antipsychotic antidepressant or antiepileptic drug comprising administering an effective amount of a known antipsychotic, antidepressant or antiepileptic drug and an effective non-toxic amount of O-(3-piperidino-2-hydroxy-1-propyl)-nicotinic amidoxime or a pharmaceutically acceptable acid addition salt thereof to the patient, wherein the administration of the O-(3-piperidino-2-hydroxy-1-propyl)-nicotinic amidoxime or a pharmaceutically acceptable acid addition salt thereof prevents or reduces the metabolic side-effect experienced by the patient requiring treatment with an antipsychotic, antidepressant or antiepileptic drug.
